(19) United States
(12) Patent Application Publication
(10) Pub. No.: US 2022/0249426 A1
(43) Pub. Date: Aug. 11, 2022

(54) INHIBITORS OF THE STING PATHWAY FOR THE TREATMENT OF HIDRADENITIS SUPPURATIVA

(71) Applicants: INSERM (INSTITUT NATIONAL DE LA SANTÉ ET DE LA RECHERCH MÉDICALE), Paris (FR); ASSISTANCE PUBLIQUE - HÔPITAUX DE PARIS, Paris (FR); UNIVERSITÉ PARIS-EST CRÉTEIL VAL DE MARNE, Creteil (FR)

(72) Inventors: Yves Levy, Creteil (FR); Sophie Hue, Creteil (FR); Jean-Louis Francette, Creteil (FR); Cindy Orvain, Creteil (FR)

(21) Appl. No.: 17/628,300

(22) PCT Filed: Jul. 23, 2020
(86) PCT No.: PCT/EP2020/070747

§ 371 Date: Jan. 19, 2022

(30) Foreign Application Priority Data
Jul. 24, 2019 (EP) 19305974.8

(51) Int. Cl. A61K 31/341; A61K 31/4045

(52) U.S. Cl. CPC A61K 31/341; A61K 31/4045

(57) ABSTRACT

Hidradenitis suppurativa (HS) is a chronic, relapsing, inflammatory skin disease in which the primary abnormality appears to affect the pilosebaceous-apocrine unit. Here, inventors' objective was to characterize the molecular mechanisms involved in the pro-inflammatory phenotype of HS-ORS cells. Transcriptomic analyses of HS-ORS cells demonstrated dysregulation of genes involved in cell proliferation and differentiation, as well as upregulation of the DNA damage response (DDR) and IFN signature. The inventors identified abnormalities in the HF-SC compartment from patients with HS, including high counts of proliferating progenitor cells and loss of quiescent bulge stem cells. Fork progression analysis revealed replicative stress responsible for ATR-CHK1 pathway activation. Accumulation of ssDNA and micronuclei in the cytosol of HS-ORS cells was found to contribute to STING activation via the DNA sensor IF116, inducing IFN synthesis independently of cGAS. STING depletion in ORS cells resulted in modulation of fork progression. These findings support the concept that, in patients with HS, impaired HF-SC homeostasis responsible for increased proliferation induces replicative stress and cytosolic ssDNA accumulation, thereby stimulating IFN synthesis through the STING pathway. Accordingly, inhibiting said pathway would be suitable of the treatment of HS.

### FIELD OF THE INVENTION

The present invention is in the field of dermatology.

### BACKGROUND OF THE INVENTION

Hidradenitis suppurativa (HS) is a chronic, relapsing, inflammatory skin disease characterized by double comedones and by recurrent, painful, deep nodules and abscesses. HS is also known as acne inversa because it does not involve the regions typically affected by acne vulgaris but instead affects sites rich in apocrine glands, including the axillae, groin, perineum, and mammary and inframammary regions (1). Patients may develop chronic inflammatory lesions with sinus tracts discharging malodorous material, cribriform scarring, and dermal fibrosis with contractures. These lesions cause severe physical and emotional distress with social embarrassment, isolation, and depression. HS is thus associated with the worst quality-of-life impairments seen in patients with common dermatoses (2). The prevalence of HS is as high as 1% of the general population in Europe (3). The many treatments used to date are generally of limited effectiveness, and recurrences are common. No formal guidelines are available for the management of HS. To develop treatments capable of improving patient outcomes, new insights into the mechanisms underlying HS are needed.

HS appears to involve a primary abnormality of the pilosebaceous-apocrine unit responsible for follicular occlusion with the secondary development of perifollicular cysts that trap commensal microbes and eventually rupture into the dermis, potentially triggering an exaggerated response of the cutaneous innate immune system (4). Abundant evidence suggests a role for chronic inflammation caused by a dysregulated immune response to bacteria and keratin filaments found ectopically in the dermis (5).

The hair follicle is a complex self-renewing appendage of the epidermis composed of an infundibulum that opens to the skin surface, sebaceous glands, and a junctional compartment between the glands and the bulge where multipotent stem cells are found (6). This hair-follicle stem-cell (HF-SC) compartment can give rise to all the epithelial cell types found in the skin, including epidermal and follicular keratinocytes, sebocytes, and hair bulb cells. Quiescent bulge stem cells are located in the outer layer of the compartment and contribute to generate the outer root sheath (ORS). ORS cells surround the hair follicle essentially as a stratified epithelium of keratinocytes that is contiguous with the epidermis. The ORS is divided into four portions, from distal to proximal: the infundibulum, bulge, sub-bulge, and lower ORS. The cells in these four regions differ in their stem-cell-associated marker expression profiles and proliferation patterns.

It was recently reported that ORS cells isolated from hair follicles of patients with HS (HS-ORS) spontaneously secrete IP10 (CXCL10) and RANTES (CCL5) (7). In HS-ORS stimulation experiments, the pattern recognition receptor (PRR) significantly increased IL-1β secretion, and IL-1β increased the production of the pro-inflammatory cytokines IL-6, IL-8, and TNF-α. These results indicated an imbalance toward a proinflammatory profile of HS-ORS cells that may explain the chronic inflammation and failure of bacterial clearance.

The cGAS-STING pathway is an important cytosolic DNA sensing pathway that activates the expression of interferons (IFNs) type I and other pro-inflammatory cytokines, thereby triggering innate immune responses to viral and bacterial DNA (8). In addition to pathogens, endogenous cytosolic DNA activates the cGAS-STING pathway in cancer cells and affects tumor development. The cGAS-STING pathway is constitutively activated in Aicardi-Goutières syndrome, which is caused by germline mutations in genes encoding factors involved in nucleic acid metabolism, such as TREX1, RNase H2 and SAMHD1 (9). The characterization of the molecular functions of these factors has shed new light on the connections between the DNA damage response (DDR) and innate immunity in disease states. Recent evidence indicates that the processing of stalled replication forks by DNA repair enzymes leads to the production of small DNA fragments that accumulate in the cytosol and activate the cGAS-STING pathway (10,11). Stalled replication forks are detected by the enzyme ATR checkpoint kinase, which binds RPA-coated ssDNA and activates the effector kinase CHK1 to prevent premature entry into mitosis and to promote the resumption of replication (12).

Other DNA sensors have been identified, such as IFN-inducible protein 16 (IFI16), DDX41, and DNA-dependent protein kinase. IFI16 is expressed in the nucleus of keratinocytes. Under inflammatory conditions, IFI16 may be recruited to STING and induce IP10 and CCL20 in response to cytosolic DNA (13). A recent study showed that DNA damage induced in keratinocytes generated an innate immune response that involved STING but not cGAS (14). This non-canonical activation of STING was mediated by IFI16 and by the DDR factors ATM and PARP-1 (14).

### SUMMARY OF THE INVENTION

As defined by the claims, the present invention relates to methods of treating hidradenitis suppurativa (HS) in patients in need thereof.

### DETAILED DESCRIPTION OF THE INVENTION

Hidradenitis suppurativa (HS) is a chronic, relapsing, inflammatory skin disease in which the primary abnormality appears to affect the pilosebaceous-apocrine unit. Here, inventors' objective was to characterize the molecular mechanisms involved in the pro-inflammatory phenotype of HS-ORS cells. Transcriptomic analyses of HS-ORS cells demonstrated dysregulation of genes involved in cell proliferation and differentiation, as well as upregulation of the DNA damage response (DDR) and IFN signature. The inventors identified abnormalities in the HF-SC compartment from patients with HS, including high counts of proliferating progenitor cells and loss of quiescent bulge stem cells. Fork progression analysis revealed replicative stress responsible for ATR-CHK1 pathway activation. Accumulation of ssDNA and micronuclei in the cytosol of HS-ORS cells was found to contribute to STING activation via the DNA sensor IFI16, inducing IFN synthesis independently of cGAS. STING depletion in ORS cells resulted in modulation of fork progression. These findings support the concept that, in patients with HS, impaired HF-SC homeostasis responsible for increased proliferation induces replicative stress and cytosolic ssDNA accumulation, thereby stimulating IFN synthesis through the STING pathway. Accordingly, inhibiting said pathway would be suitable of the treatment of HS.

Accordingly, the first object of the present invention relates to a method of treating hidradenitis suppurativa (HS) in a patient in need thereof comprising administering to the patient a therapeutically effective amount of an inhibitor of the STING pathway.

As used herein the term “hidradenitis suppurativa” has its general meaning in the art and refers to a chronic skin disease characterized by clusters of abscesses and/or cysts that most commonly affects apocrine sweat gland bearing areas. Hidradenitis suppurativa is also called acne inversa or Verneuil's disease.

In some embodiments, the method of the present invention is particularly suitable for the treatment of patients characterized by presence of HF-SC (hair-follicle stem cells) replication stress. In particular, hair-follicle stem cells of patients are characterized by the presence of at least one of the following three criteria: accumulation of cells in S phase (>25%), impaired replication fork progression, and increased proportion of cells with γ-H2AX foci (>9%). Typically said characterization may be performed as described in the EXAMPLE.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “STING pathway” refers to the pathway deciphered in the EXAMPLE and that involves the activation of STING. As used herein, the term “STING” has its general meaning in the art and refers to the adaptor protein STING (Stimulator of Interferon Genes), also known as TMEM 173, MPYS, MITA and EMS, that has been identified as a central signalling molecule in the innate immune response to cytosolic nucleic acids (Ishikawa H and Barber G N, Nature, 2008: 455, 674-678; WO2013/1666000J. Activation of STING results in up-regulation of IRF3 and NFKB pathways leading to induction of Interferon-β and other cytokines. STING is critical for responses to cytosolic DNA of pathogen or host origin, and of unusual nucleic acids called Cyclic Dinucleotides (CDNs) CDNs were first identified as bacterial secondary messengers responsible for controlling numerous responses in the prokaryotic cell. As used herein, the term “IFI16” refers to interferon-inducible protein 16 (also known as gamma-interferon-inducible protein 16, interferon-inducible myeloid differentiation transcriptional activator) and to nucleic acids, polypeptides and polymorphic variants, alleles, isoforms (e.g., those generated by alternative splicing), mutants, and interspecies homologues thereof and as further described herein. Accordingly, in some embodiments, the inhibitor of the present invention is selected among IFI16 inhibitors or STING inhibitors, or any sensor polypeptide that is involved in the activation of STING.

As used herein, the terms “antagonist” or “inhibitor” (used interchangeably herein) mean a chemical substance that diminishes, abolishes or interferes with the physiological action of a polypeptide (e.g. STING). The antagonist may be, for example, a chemical antagonist, a pharmacokinetic antagonist, a non-competitive antagonist, or a physiological antagonist, such as a biomolecule, e.g., a polypeptide, a peptide antagonist or a non-peptide antagonist. A preferred antagonist diminishes, abolishes or interferes with a physiological action of the polypeptide (e.g. STING) or activity. Specifically, an antagonist may act at the level of the interaction between a first polypeptide, e.g., STING polypeptide and a second polypeptide, for example, a binding partner. The antagonist, for example, may competitively or non-competitively (e.g., allosterically) inhibit binding of the first polypeptide e.g., STING polypeptide to the second polypeptide. A “pharmacokinetic antagonist” effectively reduces the concentration of an active drug at its site of action, e.g., by increasing the rate of metabolic degradation of the first polypeptide e.g., STING polypeptide. A “competitive antagonist” is a molecule which binds directly to the first polypeptide e.g., STING polypeptide in a manner that sterically interferes with the interaction of the first polypeptide with the second polypeptide. Non-competitive antagonism describes a situation where the antagonist does not compete directly with the binding, but instead blocks a point in the signal transduction pathway subsequent to the binding of the first polypeptide to the second polypeptide. Physiological antagonism loosely describes the interaction of two substances whose opposing actions in the body tend to cancel each other out. An antagonist can also be a substance that diminishes or abolishes expression of the polypeptide e.g., STING polypeptide. Thus, an antagonist can be, for example, a substance that diminishes or abolishes: (i) the expression of the gene encoding the polypeptide e.g., STING polypeptide, (ii) the translation of the mRNA, (iii) the post-translational modification of the polypeptide, or (iv) the interaction of the polypeptide with other polypeptides in the formation of a multi-protein complex.

Thus in some embodiments, the inhibitor is an inhibitor of expression. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of the mRNA by binding thereto and thus preventing protein translation or increasing mRNA degradation, thus decreasing the level of the polypeptide (e.g. STING), and thus activity, in a cell. For example, antisense oligonucleotides of at least about 15 bases and complementary to unique regions of the mRNA transcript sequence encoding the polypeptide (e.g. STING) can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. Gene expression can be reduced by contacting a patient or cell with a small double stranded RNA (dsRNA), or a vector or construct causing the production of a small double stranded RNA, such that gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. In a particular embodiment, the endonuclease is CRISPR-cas. In some embodiment, the endonuclease is CRISPR-cas9, which is from Streptococcus pyogenes. The CRISPR/Cas9 system has been described in U.S. Pat. No. 8,697,359 B1 and US 2014/0068797. In some embodiment, the endonuclease is CRISPR-Cpf1, which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor is a small molecule such as a small organic molecule, which typically has a molecular weight less than 5,000 kDa. Examples of STING inhibitors are described in WO2015185565 as well as in U.S. Pat. No. 9,549,944B2.

In some embodiments, STING inhibitors are selected from the compounds described in Haag S. M. et al., 2018. Targeting STING with covalent small-molecule inhibitors. Nature 559:269-73.

In particular, the STING inhibitor is N-(4-iodophenyl)-5-nitrofuran-2-carboxamide, also known as C-176.

In some embodiment, the STING inhibitor is N-(4-Ethylphenyl)-N′-1H-indol-3-yl-urea also known as H-151 that has the formula of:

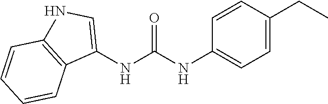 [SMILES: CCc1ccc(NC(=O)Nc2c[nH]c3ccccc23)cc1]

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumour size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg.

Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). In some embodiments, the inhibitor of the present invention is administered topically. The inhibitor of the present invention is used or applied on lesion area(s) of the skin, and preferably also around lesion area(s) and/or on area(s) suspected to become lesion areas. By “lesion”, “skin lesion” or “lesion area of the skin”, it is herein meant a painful, itching, inflamed and/or infected area of the skin, preferably at least an inflamed and/or infected area of the skin. An area suspected to become a lesion area is for example a skin area of the axillary, inguinal, under breast, anal and/or genital, back or hair region. In some embodiments, the inhibitor of the present invention is used or administered topically on axillary, inguinal, under breast, anal and/or genital region(s). In some embodiments, the inhibitor of the present invention is used, administered or applied one to three times per day. In some embodiments, the inhibitor of the present invention used or administered at least until the symptoms of the disease disappear, for example at least until the lesions disappear. In some embodiments, the inhibitor of the present invention is used or administered several days or several weeks after the disappearance of symptoms of the disease, for example until the lesions disappear, possibly with a gradual reduction in the frequency of administration of said inhibitor of the present invention.

Typically the inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils.

In some embodiments, as stipulated above, it may be desirable to administer the agent of the present in a topical formulation. As used herein the term “topical formulation” refers to a formulation that may be applied to skin. Topical formulations can be used for both topical and transdermal administration of substances. As used herein, “topical administration” is used in its conventional sense to mean delivery of a substance, such as a therapeutically active agent, to the skin or a localized region of a subject's body. As used herein, “transdermal administration” refers to administration through the skin. Transdermal administration is often applied where systemic delivery of an active is desired, although it may also be useful for delivering an active to tissues underlying the skin with minimal systemic absorption. Typically, the topical pharmaceutically acceptable carrier is any substantially nontoxic carrier conventionally usable for topical administration of pharmaceuticals in which the inhibitor of the present invention will remain stable and bioavailable when applied directly to skin surfaces. For example, carriers such as those known in the art effective for penetrating the keratin layer of the skin into the stratum comeum may be useful in delivering the inhibitor of the present invention to the area of interest. Such carriers include liposomes. Inhibitor of the present invention can be dispersed or emulsified in a medium in a conventional manner to form a liquid preparation or mixed with a semi-solid (gel) or solid carrier to form a paste, powder, ointment, cream, lotion or the like. Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy. The topical acceptable carrier will be any substantially non-toxic carrier conventionally usable for topical administration in which inhibitor of the present invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Any suitable carrier or vehicle effective for topical administration to a patient as know in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. Topical acceptable carriers may be similar or identical in nature to the above described topical pharmaceutically acceptable carriers. It may be desirable to have a delivery system that controls the release of inhibitor of the present invention to the skin and adheres to or maintains itself on the skin for an extended period of time to increase the contact time of the inhibitor of the present invention on the skin. Sustained or delayed release of inhibitor of the present invention provides a more efficient administration resulting in less frequent and/or decreased dosage of inhibitor of the present invention and better patient compliance. Examples of suitable carriers for sustained or delayed release in a moist environment include gelatin, gum arabic, xanthane polymers. Pharmaceutical carriers capable of releasing the inhibitor of the present invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the inhibitor of the present invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The inhibitor of the present invention can also be administered in combination with other pharmaceutically effective agents including, but not limited to, antibiotics, other skin healing agents, and antioxidants. In some embodiments, the topical formulation of the present invention comprises a penetration enhancer. As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

The invention will be further illustrated by the following figures and examples. However, these examples and figures should not be interpreted in any way as limiting the scope of the present invention.

### FIGURES

FIG. 1: IFI16-STING pathway induces interferon (IFN) production in outer root sheath (ORS) cells from patients with hidradenitis suppurativa (HS). (A) Relative mRNA levels of IFN-β and IP10 in HS-ORS and HD-ORS cells. **P<0.01; ns, nonsignificant; Mann-Whitney rank-sum test. (B) Nonparametric Spearman correlation analysis of the expression of IFN-β genes and percentage of cells in phase S in HS-ORS cell populations. (C) Levels of IFN-β mRNA in HS-ORS (n=10) and HD-ORS (n=4) cell populations transfected with siSTING for 2 days before IFN-β mRNA quantification. (D) IFN-β mRNA levels in HS-ORS cell populations (n=3) transfected with siRNA-cGAS or siRNA-IFI16 for 2 days before IFN-β mRNA quantification. (E) DNA fiber spreading analysis of fork progression in HS-ORS cell populations (n=2) transfected with siSTING for 2 days before DNA fiber spreading. ****P<0.0001, Mann-Whitney rank-sum test. (F) Western blot analysis of the levels of phospho-IRF3 in ORS transfected with siSTING or siCtrl. Quantification of phospho-IRF3 signal intensity in HS-ORS (n=7) and HD (n=5).

FIG. 2: Outer root sheath (ORS) cells from two patients were treated with two drugs (A. Ruxolitinib B. H151) targeting the interferon (IFN) pathway. mRNA levels of MX1, IP10, IFI27 and OAS1 in ORS. HS-ORS (n=2) were treated with two drugs during 24 hours before performing mRNA quantification. Red line showed DMSO level expression for each gene

For the first patient, ORS treated with the drug B expressed reduced levels of MX1, IP10, IFI27 and OAS1 transcripts compared to ORS treated with DMSO. Drug A gave the same profile except for OAS1 transcripts. In patient 2, a decrease of ISGs transcripts was observed in ORS treated with the drug A. Altogether, these preliminary results suggest that targeting INF pathway reduced the level of inflammation in HS-ORS.

### EXAMPLE

Material & Methods:

Participants and Samples

Skin samples were collected at the dermatology and plastic surgery department of the Henri Mondor university hospital during unroofing of axillary or perineal lesions in 33 patients with HS and during brachioplasty or abdominoplasty in 25 healthy individuals. Hair-rich skin sites were collected from the surgical specimens and processed as described by Aasen T (6,15) in order to obtain ORS cells.

The 33 patients with HS had a mean age of 30.4 years (range, 14-71 years) and a mean body mass index of 27.1 kg·m⁻²; 18 (54%) were women and 12 (36.3%) were smokers. The Hurley stage was I in one patient, II in nine patients, and III in 23 patients. None of the patients used topical treatments or took immunosuppressants (Table 1). The 25 controls had a mean age of 34.5 years (range, 19-57 years); 21 were women and 4 were men. None had a history of skin disease or malignancy.

TABLE 1
Patient characteristics

| Parameters | Patients (n = 33) | |
|---|---|---|
| Age, years, mean (range) | 30.4 | (14-71) |
| Females, n (%) | 18 | (54%) |
| Body mass index, | 27.1 | |
| kg/m², mean | | |
| Smokers, n (%) | 12 | (36.3%) |

| Hurley stage | |
|---|---|
| I | 1 |
| II | 9 |
| III | 23 |
| LC phenotype | |
| 1 | 27 |
| 2 | 4 |
| 3 | 2 |
| Treatments | |
| Immunosuppressants | 0 |
| Antibiotics | 33 |
| Associated diseases | |
| Spondyloarthritis | 1 |
| Crohn's disease | 1 |

Outer Root Sheath (ORS) Cell Culture

When ORS cells were sub-confluent, the feeder layer was removed with PBS-EDTA (0.71 mM) and the ORS cells were detached after incubation with trypsin (TrypLe Express 1×, Life Technologies, Carlsbad, Calif.). The cells were seeded in defined medium without fetal calf serum (FCS) (Epilife, Life Technologies) or in complete DMEM F12 medium, on irradiated 3T3 feeder layers. Experimental procedures were done at P1/2, except 53BP1 analysis and IP10/IFNβ mRNA quantification, which were performed at P 3/4.

Microarray Sample Preparation

Total RNA was purified from cells using RNeasy Plus Micro Kit (Qiagen, Hilden, Germany), and globin mRNAs were removed using the GLOBINclear-Human Kit (Ambion, Foster City, Calif.). The RNAs were quantified with the Quant-iT RiboGreen RNA Assay Kit (Thermo Fisher Scientific, Waltham, Mass.) and their quality was then controlled using the Agilent Bioanalyzer System (Agilent, Santa Clara, Calif.). In vitro RNA transcription was obtained using the Ambion Illumina TotalPrep RNA Amplification Kit (Applied Biosystems/Ambion, Saint Aubin, France). Labelled cRNA was hybridized onto Illumina Human HT-12v4 BeadChips (Illumina, San Diego, Calif.). Quality controls were processed using GenomeStudio software (Illumina). Differentially expressed genes were identified using quantile normalized data as input to gene-specific analysis (GSA) (Partek Flow software, Partek, Chesterfield, MI). Hierarchical clustering was performed using the Euclidean distance method. Only genes with adjusted P values (False Discovery Rate)≤0.2 and a fold change≥1.5 were classified as differentially expressed. Functional enrichment analysis of differentially expressed genes was with Ingenuity Pathway software (Ingenuity Systems, Redwood City, Calif.).

Clonal Expansion

Clonal expansion was performed by seeding 5000 ORS cells at P1 onto 0.5·10⁶ irradiated 3T3-J2 cells in a feeder layer, in 60-mm Petri dishes. After 14 days of culturing, the ORS cells were fixed with formalin for 10 minutes and immediately stained with rhodamine for 20 minutes. The Petri dishes were washed and left to dry. The ORS cell colonies were then examined and counted with Image J software.

Small Interfering RNA (siRNA) Transfection

The following siRNAs were used: siSTING (CUGCAUCCAUCCAUCCCGUdTdT; SEQ ID NO:1, Sigma #Hs01_00031038, Sigma, St Louis, Mich.), siIFI16 (L-020004-00-0005, ON-TARGETplus Human IFI16 siRNA SMARTpool, Horizon Discovery, Waterbeach, UK), and sicGAS (L-015607-02-0005, ON-TARGETplus Human MB21D1 siRNA SMARTpool, Horizon Discovery). All siRNAs were used at a final concentration of 10 μM.

ORS cells were seeded in defined medium without FCS (Epilife, Life Technologies) at P2/3 and transfected with siRNA at P3/4, twice on two consecutive days (days 0 and 1), using INTERFERin (PolyPlus Transfection, Illkirch-Graffenstaden, France) according to the manufacturer's protocol. The ORS cells were lysed 48 h after transfection to allow measurement of protein and mRNA expression.

Cell Cycle Analysis

ORS cells were grown successively at P2 and P3 in defined medium without FCS (Epilife, Life Technologies). Once the cells were half-confluent, they were fixed in ice-cold PBS/70% ethanol then resuspended in FxCycle™ PI/RNase Staining Solution (Thermo Fisher Scientific) according to the manufacturer's protocol and incubated for 30 minutes in the dark at room temperature before fluorescence-activated cell sorting (FACS) analysis.

Hair Follicle Phenotype Analysis

Hair-follicle cells obtained after skin dissociation were stained with the LIVE/DEAD™ Fixable Aqua Dead Cell Stain Kit (Thermo Fisher Scientific) then fixed with Foxp3/Transcription Factor Staining Buffer (eBioscience, Thermo Fisher Scientific). The cells were stained with primary mouse anti-human cytokeratin 15 (LHK15, Thermo Fisher Scientific) and secondary FITC Goat anti-Mouse Ig (BD Biosciences, Franklin Lakes, N.J.) antibodies. Surface staining was then performed using a mix of the following antibodies: PE mouse anti-human CD34 (8G12, BD Biosciences), APC-H7 mouse anti-human CD45 (2D1, BD Biosciences), Pe-Cy7 mouse anti-human CD117 (104D2, BD Biosciences), BV421 mouse anti-human CD200 (MRC OX-104, BD Biosciences), and PerCp-Cy5.5 rat anti-human CD49f (GoH3, BD Biosciences). The cells were run on an LSRII flow cytometer (BD Biosciences) and analyzed with FlowJo software, version 10.2 (FlowJo LLC, Ashland, Oreg.).

RNA Extraction, Reverse Transcriptase (RT) Reaction, and Quantitative Polymerase Chain Reaction (qPCR)

ORS cells were lysed with RLT Plus Buffer (Qiagen). The RNA was extracted using the RNeasy Plus Mini Kit (Qiagen) according to the manufacturer's protocol then converted into cDNA using the QuantiTect Reverse Transcription Kit (Qiagen) for IFNβ detection and the High-Capacity cDNA Reverse Transcription Kit (Applied Biosystems, Thermo Fisher Scientific) for the expression of other genes.

For real-time PCR analysis of the cDNA samples thus obtained, we prepared custom-made primer sets using the Brilliant II SYBR green QPCR master mix (Agilent Technologies) or Quantitect SYBR Green PCR Kit (Qiagen) according to the manufacturers' protocols. Real-time PCR was performed on a MX3000P device (Agilent). The GAPDH or OAZ1 mRNA level was used for normalization. The relative expression (ΔCt) of mRNAs of each gene was computed using GAPDH or OAZ-1 expression as the reference.

Western Blotting

Lysates were resolved by SDS-PAGE and the gels were transferred using the iBlot device (Thermo Fisher Scientific). After incubation with PBS-Tween 0.1%-5% non-fat dry milk for 2 hours, the membranes were incubated with the primary antibody overnight at 4° C. according to the manufacturer's protocol then with the secondary antibodies in PBS-Tween 0.1%-5% non-fat dry milk for 1 hour at room temperature. Revelation was by chemiluminescence with SuperSignal™ West Femto Maximum Sensitivity Substrate (Thermo Fisher Scientific).

DNA Fiber Spreading

DNA fiber spreading was performed as described by Jackson and Pombo (16). Briefly, subconfluent ORS cells were labelled sequentially with 10 μM 5-iodo-2′-deoxyuridine (IdU) then with 100 μM 5-chloro-2′-deoxyuridine (CIdU) for 30 minutes. The cells were loaded onto a glass slide (StarFrost, Lowestoft, UK), lysed with spreading buffer (200 mM Tris-HCl pH 7.5, 50 mM EDTA, 0.5% SDS), and slowly spread down the slides, which were then fixed in 3:1 methanol:acetic acid for 10 min and allowed to dry. Slide immunostaining was with mouse anti-BrdU to detect IdU, rat anti-BrdU to detect CIdU, and corresponding secondary antibodies conjugated to various Alexa Fluor dyes (Thermo Fisher Scientific). Nascent DNA fibers were visualized by immunofluorescence microscopy (ApoTome, Zeiss Oberkochen, Germany). The DNA fiber images thus acquired were analyzed using MetaMorph Microscopy Automation and Image Analysis Software (Molecular Devices, San Jose, Calif.) and the results were evaluated statistically by applying the two-sided Mann-Whitney rank-sum test with GraphPad Prism software (GraphPad, La Jolla, Calif.).

Immunohistochemistry

To assess cell proliferation in the epidermis and hair follicles, we performed Ki-67 staining on 3-μm thick sections of five skin samples with HS lesions and four samples from axillary scars of age-matched patients with HS. After antigen retrieval at pH 6, we applied rabbit anti-Ki-67 clone SP6 (Zytomed Systems, Berlin, Germany) diluted 1/50, using the BOND-III device (Menarini-Leica, Florence, Italy and Wetzlar, Germany) with diaminobenzidine chromogen.

Immunocytochemistry

Total hair-follicle cells were cytocentrifuged for 5 minutes at 600 g and dried overnight. Cytospins were fixed in 4% paraformaldehyde (PFA) in phosphate-buffered saline (PBS) and kept at −20° C. until use. When ORS cells seeded on culture slides became subconfluent, they were fixed in 4% PFA in PBS and kept at −20° C. until use. The cells were permeabilized then blocked as described in Supplementary Table S2, incubated with the primary antibody in a wet chamber, washed with PBS-0.1% Tween 20 for 5 minutes three times, and incubated with the secondary antibody. After three 5-minute washes with PBS-0.1% Tween 20, the slides were mounted using ProLong Gold antifade with DAPI (Thermo Fisher Scientific).

Statistics

Groups were compared using the two-sided Mann-Whitney U test. Spearman's rank test was applied to assess bivariate correlations, and linear regression analysis was performed to produce an accompanying best-fit line. All statistical analyses were performed using GraphPad Prism (version 7.0, GraphPad Software, La Jolla, Calif.).

Results

Transcriptomic Analyses of HS-ORS Cells Revealed an Interferon (IFN) Signature and Cell-Cycle Pathway Abnormalities

To identify the mechanisms involved in the pro-inflammatory phenotype of HS-ORS cells, we isolated ORS cells from hair follicles and performed transcriptomic analyses after ORS cells amplification at passage (P) 3. The use of principal component analysis to compare the gene expression profile thus obtained between HS-ORS samples (n=6) and healthy donor samples (HD-ORS, n=5) revealed significant differences (data not shown). The HS-ORS samples were characterized by dysregulation of genes involved in cell proliferation and differentiation and by upregulation of the DDR and cell-cycle G2/M checkpoint pathways (data not shown). Of note, in the HS-ORS samples, we observed induction of genes involved in the insulin-like growth factor 1 (IGF1) pathway, which plays important roles in hair-follicle development and cycling (6); and induction of genes strongly associated with the IFN signature, with overexpression of IFN-stimulated genes and genes encoding IFN regulatory factors (IRFs) (data not shown). To confirm this in vitro IFN signature, we assessed expression of ISGs by RT-qPCR in freshly total isolated hair follicle cells. We observed an overexpression of IP-10, IFI27 and OAS1 in HS-ORS compared to HD-ORS (IP-10: 12.65±15.34 versus 0.4833±0.37, p=0.0006; IFI27: 53.45±47.34 versus 19.71±6.675, p=0.0048; OAS1: 5.409±2.54 versus 2.658±1.32, p=0.019, respectively; data not shown). MX-1 mRNA expression was increased in HS-ORS compared to HD-ORS although the difference was not statistically significant between HS patients and HD (2.059±1.74 versus 0.8533±0.75, p=0.09). These findings indicate that ex vivo total hair follicle cells display type I IFN signature.

Hair-Follicle Stem Cells (HS-SCs) from Patients with HS Lacked the Quiescent Bulge Stem-Cell Population

To confirm the findings from the transcriptomic analysis of cell proliferation, we performed Ki67 staining of samples from HS skin lesions. Compared to normal skin samples, the HS samples contained higher counts of Ki67-positive keratinocytes and hair-follicle cells (data not shown). Ki67-positive keratinocytes were prominent in the epidermis overlying infiltrates and in the sebaceous glands.

We then performed a colony-forming efficiency assay to assess ORS cell proliferation rates. At the initial concentration of 5000 cells per well, colony-forming was far more efficient with HS-ORS cells than with HD-ORS cells (data not shown). Moreover, colony number and size were far greater with the HS-ORS samples (n=3) than with the HD-ORS samples (n=3). Thus colony size distribution was as follows: <1 mm², 60 versus 62; 1-10 mm², 75 versus 64; 10-100 mm², 22 versus 3; and >100 mm², 4 versus 0; respectively.

HF-SCs are a heterogeneous population with marked variations in cell-cycle dynamics. To better characterize the HF-SC compartment, we used flow cytometry to investigate the phenotype of freshly isolated hair-follicle cells. Only CD45⁻ CD117⁻ cells in the starting population were analyzed; we thus excluded CD45⁺ hematopoietic cells and CD117⁺ melanocytes. Using the biomarker combinations determined by Inoue et al. (17), we defined cells from the basal bulge as CD200⁺CD34⁻, from the upper sub-bulge as CD200⁺CD34⁺, and from the sub-bulge as CD200⁻CD34⁺(data not shown). Compared to HD samples, HS samples were characterized by higher proportions of sub-bulge and upper sub-bulge cells (sub-bulge, 56.5% versus 22.35%, P=0.0029; upper sub-bulge, 6.17% versus 1.64%, P=0.0059) (data not shown). In line with these results, bulge cell depletion was noted in HS compared to HD samples (3.39% versus 12.70%, P=0.0059) (data not shown). When we classified the basal bulge cells into two populations based on K15 and CD49f expression (data not shown), we found that the CD200⁺CD34⁻K15^{high}CD49f^{high} bulge cells were in a quiescent state (G0/G1), whereas the CD200⁺CD34⁻K15^{low}CD49f^{low} bulge population contained a substantial number of dividing cells in S— and G2/M phase (data not shown). Importantly, the well-defined quiescent CD200⁺CD34⁻K15^{high}CD49f^{high} bulge population was absent in 7 of 11 patients with HS (data not shown). These findings are consistent with loss of quiescent stem cells and increased cell proliferation in patients with HS.

Spontaneous Activation of the ATR-CHK1 Pathway in HS-ORS

As proliferating ORS cells were found in increased numbers in patients with HS, we investigated their cell-cycle distribution after in vitro amplification (data not shown). Compared to the HD-ORS population, the HS-ORS population contained a higher percentage of S-phase cells (20.5%±4.2% versus 16.3%±3.4%, P=0.0028) (data not shown). In line with this result, the percentage of cells in G0/G1 was lower in the HS-ORS population compared to the HD-ORS population (62.1%±5.2% versus 67.1%±4.1%; P=0.021). The percentage of cells in G2/M phase varied from 5% to 24% in the HS-ORS population and was fairly stable at about 15% in the HD-ORS population (data not shown).

We investigated whether the higher percentage of S-phase cells in patients with HS was related to increased replication stress, which is defined as a global perturbation of the DNA replication program that alters replication fork speed and activates the ATR-CHK1 pathway (18). We monitored the progression of individual forks by DNA fiber spreading after labeling ongoing DNA synthesis in HD-ORS and HD-ORS cells with two successive pulses of the thymidine analogs IdU and CIdU (19). In all three HD-ORS samples, IdU track length was about 5 μm (data not shown). Of the 6 HS-ORS samples, 5 showed a considerably longer track length, indicating severely impaired replication fork progression.

We then investigated whether the ATR-CHK1 pathway was spontaneously activated in HS-ORS cells. First, we monitored the phosphorylation of the histone variant H₂AX by immunohistochemistry in ORS-cell DNA amplified in vitro. Phosphorylation of γH₂AX was greater in the HS-ORS than in the HD-ORS samples, but the difference was not statistically significant (data not shown). We then monitored the phosphorylation of the DNA replication checkpoint CHK1 and of the DNA damage checkpoint CHK2 by Western blotting after in vitro amplification. CHK1 phosphorylation was significantly greater in the HS-ORS than in the HD-ORS samples (HS phosphoCHK1/CHK1 0.53 versus HD phosphoCHK1/CHK1 0.025, P=0.003) (data not shown). In contrast, CHK2 phosphorylation was not significantly different between the two populations (data not shown). These results indicate spontaneous replication stress responsible for ATR-CHK1 pathway activation in HS-ORS cells.

Incomplete DNA synthesis during the S phase leads to formation of 53BP1 nuclear bodies in the subsequent G1 phase. Using immunocytochemistry, we found greater accumulation of 53BP1 foci in HS-ORS than in HD-ORS samples (19.3 versus 9.13, P=0.036, data not shown).

To confirm these in vitro data, we used immunohistochemistry to assess CHK1 phosphorylation in freshly isolated hair-follicle cells from patients with HS and from healthy controls. The proportion of CHK1-phosphorylated cells was higher in the HS group (15.8% versus 4.9%, P=0.0065; data not shown). The CHK1-phosphorylated cells were negative for the leukocyte marker CD45 and positive for the HF-SC marker cytokeratin-15 (data not shown). These cells were negative for CD49f and CD34, two markers expressed by the lower ORS (17) (data not shown). Together, these data indicate that ex vivo HF-SCs, but not hematopoietic cells, from patients with HS constitutively exhibit a replication stress profile.

Presence of Micronuclei and Cytosolic ssDNA in HS-ORS Cells

DNA damage is associated with increased formation of micronuclei, whose rupture exposes DNA to PRRs and activates the STING pathway. To detect micronuclei, we stained ORS cells for lamin B1, which is an integral nuclear envelope protein and therefore a reliable marker for micronuclei. The proportion of micronuclei positive cells was higher in the HS-ORS than in the HD-ORS samples (4.26% versus 2.27%, P<0.05) (data not shown).

Nuclear DNA damage can also result in ssDNA being present in the cytosol, via a poorly understood process (20). Using immunofluorescence microscopy, we detected ssDNA in the cytosol of HS-ORS cells (n=6; data not shown) but not HD-ORS cells (n=3). This signal decreased after HS-ORS cell treatment with 51 nuclease, confirming its specificity (data not shown). Interestingly, accumulation of ssDNA was not found in the cytosol of interfollicular keratinocytes from the same patients with HS (n=3; data not shown). These results suggest that the accumulation of cytosolic ssDNA in patients with HS may be specific of the ORS.

Expression of Pro-Inflammatory Type I Interferons Via the STING Pathway in HS-ORS Cells

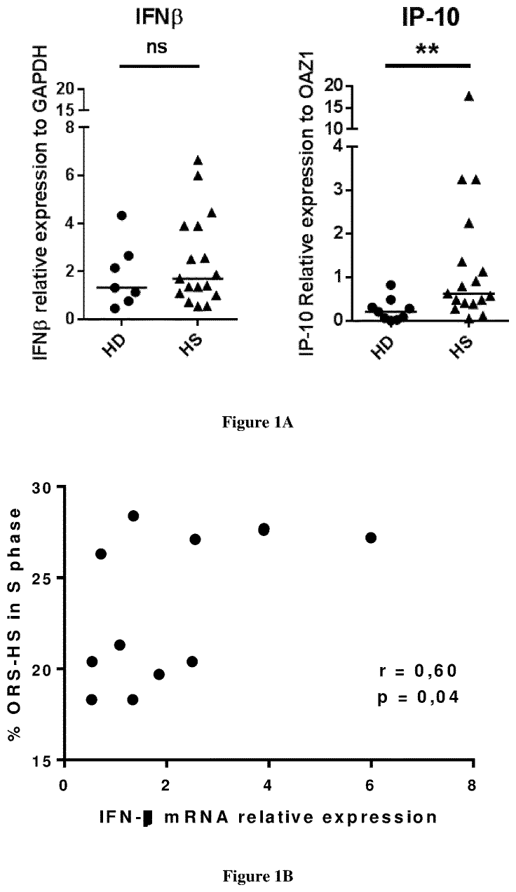

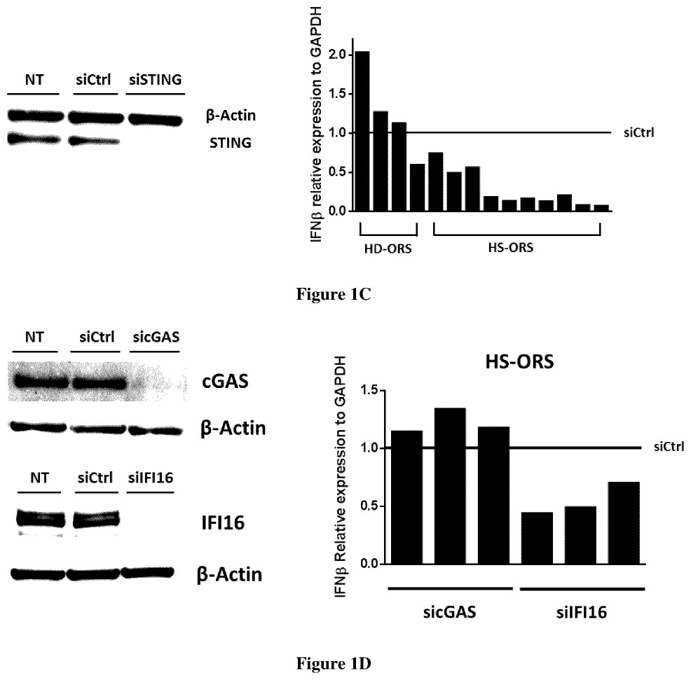

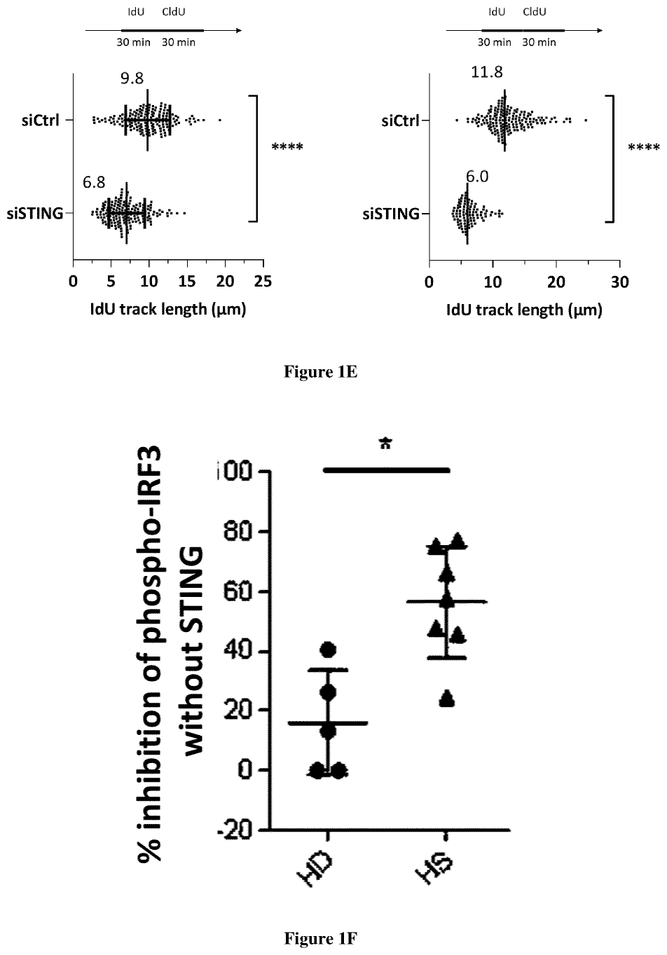

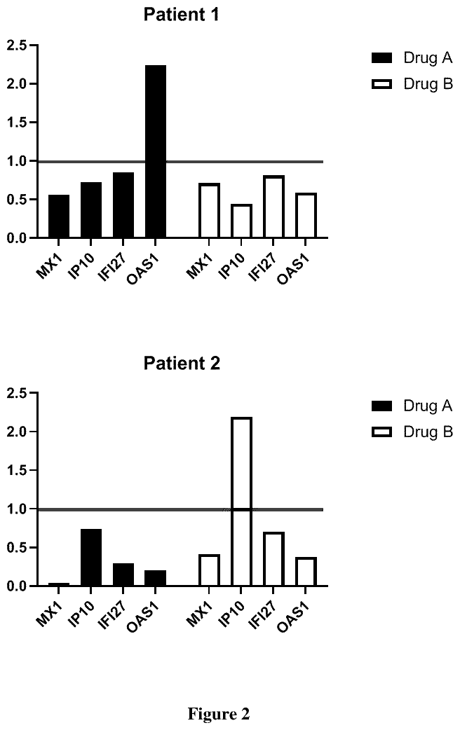

Because cytosolic DNA fragments and damaged micronuclei activate the STING pathway to induce expression of pro-inflammatory IFN type I, we assessed the expression of IFN-stimulated genes, using quantitative reverse-transcription PCR. The results showed IP-10 overexpression in HS-ORS cells compared to HD-ORS cells (0.69 versus 0.29 AU, P=0.029; FIG. 1A). IFN-β mRNA expression was increased in HS-ORS compared to HD-ORS cells, although the difference was not statistically significant (1.77 versus 1.72, P=0.584). However, the levels of IFN-β mRNA correlated positively with the percentage of cells in S phase (r=0.6035, P=0.0413) (FIG. 1B).

As IFN-β is the first target gene in STING pathway activation, to test whether the DNA sensing adaptor STING was involved in IFN type I production we transfected ORS cells with STING siRNA (siSTING) then evaluated the IFN-β transcripts. HS-ORS cells lacking STING expressed lower IFN-β transcript levels compared to HS-ORS cells transfected with a scramble siRNA (FIG. 1C). By contrast, absence of STING was not associated with a difference in IFN-β transcript levels in HD-ORS cells. We also assessed OAS1 and IP-10 as ISGs in ORS lacking STING. In HS patients, a decrease of ISGs transcripts was observed between ORS lacking STING and ORS transfected with a scramble siRNA. As expected, no change in ISGs transcripts was noted in absence of STING in ORS from HD. STING activates transcription factor IRF3. Western blot analysis of IRF3 phosphorylation at Ser396 (pIRF3) reveals an inhibition of pIRF3 in HS-ORS. In healthy donors, no modification of pIRF3 was observed between HD-ORS lacking STING and HD-ORS transfected with a scramble siRNA (FIG. 1F).

Next, we assessed whether cGAS was also required for IFN type I production. Depletion of cGAS had no effect on IFN-β transcript levels in HS-ORS cells, suggesting that the classical cGAS-STING pathway was not involved in ssDNA recognition (FIG. 1D). Since the non-canonical STING activation pathway is mediated by the DNA binding protein IFI16, we evaluated IFI16 involvement in IFN type I production by HS-ORS cells. HS-ORS cells lacking IFI16 expressed lower IFN-β transcript levels compared to HS-ORS cells transfected with a scramble siRNA (FIG. 1D). We observed a heterogeneous response for the others tested ISGs, suggesting that others sensors may be involved in STING activation. However, IRF3 phosphorylation in HS-ORS depleted with IFI16 was decreased in all cases compared to a scramble siRNA (data not shown). We observed an increased pool of IFI16 localizing to the cytoplasm in HS-ORS compared to HD-ORS (5.1±1.4% versus 3.1±1.7%; p=0.02) consistent with a role in the detection of cytoplasmic DNA (data not shown). Lastly, we noticed a decreased of cGAS protein expression in ORS lacking IFI16 (data not shown) suggesting that cGAS and IFI16 cooperate to form a DNA receptor complex as observed in human macrophages. These data suggested that cytosolic ssDNA in HS-ORS cells induced IFN type I production via the STING pathway, and more particularly IFI16-STING pathway.

Finally, we investigated whether the faster replication fork progression observed in most patients with HS involved constitutive activation of the STING pathway. Remarkably, we found that the IdU track length in both HS-ORS cell populations studied was decreased to the length observed in HD-ORS cells after STING depletion (FIG. 1E). These results suggest that STING may modulate replication fork progression.

### DISCUSSION

HS is a common disease in which the primary abnormality, which remains unelucidated, involves the pilosebaceous-apocrine unit. Here, we identified major homeostatic abnormalities of the HF-SC compartment in patients with HS. Clonotypic analysis and HF-SC characterization demonstrated an increased number of proliferating progenitor cells and loss of quiescent stem cells associated with spontaneous replication stress in patients with HS compared to healthy donors. HS-ORS cells were characterized by accumulation of cytosolic ssDNA and micronuclei and by the induction of IFN synthesis through the STING pathway. To our knowledge, such alterations of the HF-SC compartment have not been described in other diseases of the hair follicle. In reversible types of alopecia such as alopecia areata, the inflammatory process targets hair-follicle progenitor cells but spares HF-SCs (21), whereas a defect in HF-SC conversion to progenitor cells plays a role in the pathogenesis of androgenetic alopecia (22). Our results suggest that the lack of quiescent stem cells may be due to increased stem-cell differentiation and not to HF-SC destruction as observed in scarring alopecia associated with permanent hair loss (23).

Another original finding from our study is that cell homeostasis impairments led to DNA damage and were associated with replication stress, as shown by alterations in replication fork progression, activation of the ATR-CHK1 pathway, and accumulation of cytosolic ssDNA. These alterations were specific of HS-ORS cells: we did not observe CHK1 phosphorylation in hematopoietic cells or ssDNA accumulation in the cytosol of interfollicular keratinocytes from patients with HS. They suggest a pathophysiological process that tipped the balance toward DDR control by intrinsic cell properties rather than by the local microenvironment. However, a self-perpetuating mechanism secondary to local inflammation and responsible for the production of reactive oxygen species that may also contribute to genotoxicity and genomic instability cannot be completely ruled out. Globally, our observation is reminiscent of data obtained recently in mice (24) and showing that multipotent bulge HF-SCs were more resistant to DNA damage-induced cell death compared to other epidermal cells, because they expressed higher levels of the anti-apoptotic protein Bcl2 (25). Moreover, bulge HF-SCs display efficient DNA repair and enhanced non-homologous end-joining (NHEJ) activity (24), which are more error prone than homology-mediated double-strand break repair pathways. Conceivably, greater resistance of human HF-SCs to DNA-damage-induced apoptosis and increased NHEJ activity may allow the accumulation of mutations, thereby increasing genomic instability.

Recent research has provided sound evidence that genomic DNA damage triggers an inflammatory response through the accumulation of cytosolic DNA fragments and the activation of STING (11, 20). Cytosolic DNA is either generated by aberrant DNA repair activities or by the rupture of micronuclei, which form during mitosis as a consequence of broken or lagging chromosomes (10, 26, 27). Interestingly, we found both micronuclei and ssDNA fragments in the cytosol of HS-ORS cells. The increased numbers of micronuclei in HS-ORS cells may result from greater replication stress interfering with proper chromosome segregation. Alternatively, HF-SCs may display increased NHEJ activity (26), which is an error-prone DNA repair pathway that generates dicentric chromosomes. Cytosolic DNA is normally degraded by the enzyme TREX1 exonuclease, whose loss is associated with chronic IFN induction (8). We suggest that, under conditions of replication stress, chromosome missegregation induced by either replication completion defects or NHEJ-mediated chromosome rearrangements may promote the accumulation of cytosolic DNA, eventually overwhelming the buffering capacity of TREX1.

In keeping with other reported pathophysiological mechanisms, our results showed that endogenous cytosolic DNA activated the STING pathway to induce IFN type I production in ORS cells. Excessive cGAS-STING-dependent IFN type I release has been implicated in both myocardial infarction (28) and interstitial lung disease (29). Genetic deficiencies that compromise DDR functions also induce IFN and lead to autoinflammatory diseases such as the immunodeficiency syndrome ataxia-telangiectasia due to ATM mutations (29). Similarly, Aicardi-Goutières syndrome is driven by chronic IFN signaling caused by a recessive mutation in one of a few genes involved in nucleic acid metabolism.

We identified IFI16 as a DNA sensor, whereas cGAS was not required for the STING-mediated IFN type I response of HS-ORS cells. IFI16 and cGAS cooperate to activate STING during DNA sensing in human keratinocytes (30). Human keratinocytes do not normally respond to cytosolic DNA by mounting an innate immune response. The redistribution of a small pool of cellular IFI16 from the nucleus to the cytosol is critical to DNA sensitivity stimulation in keratinocytes (13), and TNF treatment is associated with IFI16 accumulation in the cytosol. A non-canonical STING pathway inducing IFN type I production in response to DNA damage has been reported in keratinocytes and may act in parallel with the cGAS-STING pathway to signal genotoxic stress (14). Conceivably, inflammation may cause IFI16 to relocate to the cytosol, where it may bind ssDNA, thereby activating the STING pathway.

Our findings may have important clinical implications. First, as discussed above, our results are consistent with a vicious circle that is triggered by HF-SC replicative stress and leads to chronic inflammation. Second, they are reminiscent of the mechanisms involved in Notch signaling pathway defects, which are the most common genetic susceptibility factors reported in patients with familial HS (31). In several manipulated cell-line models characterized by Notch pathway inactivation, keratinocyte proliferation and differentiation were severely impaired (32,33). Notch signaling is also required for postnatal hair-cycle homeostasis, because it maintains normal HF-SC proliferation and differentiation (34). Third, generation of ORS seems to be a useful and relevant model for investigating the pathophysiology of HS. Our transcriptomic analysis of these cells revealed pathways similar to those previously reported by our group and others, with enrichment in genes involved in keratinocyte differentiation, epidermis development pathways (7,35), and upregulation of IFN pathways (36,37) in whole skin isolated from HS lesions. Finally, recent studies identified phenotypic heterogeneity in patients with HS, suggesting that pathogenic pathways may also vary across patient subsets. Our results may help to separate two different patient subsets characterized by the presence or absence of HF-SC replication stress. We suggest defining replication stress as the presence of at least one of the following three criteria: accumulation of cells in S phase (>25%), impaired replication fork progression, and increased proportion of cells with γ-H2AX foci (>9%). It is worth noting that 6 of the 8 patients with replication stress but only 3 of 10 patients without replication stress were males. However, the number of patients is too small for definitive conclusions. Further work is needed to determine whether replication stress can serve to stratify patients and identify new treatment targets.

We used two drugs targeting IFN pathway. HS-ORS (n=2) were treated with drugs during 24 hours before performing mRNA quantification. Four ISGs: MX1, OAS1, IFI27 and IP10 were analyzed. ORS treated with either the drug A or B expressed reduced levels of MX1, IP10, IFI27 and OAS1 transcripts compared to ORS treated with DMSO suggesting that targeting INF pathway reduced the level of inflammation in HS-ORS.

### REFERENCES

Throughout this application, various references describe the state of the art to which this invention pertains. The disclosures of these references are hereby incorporated by reference into the present disclosure.
1. Revuz, J. Hidradenitis suppurativa. J Eur Acad Dermatol Venereol 23, 985-998 (2009).
2. Matusiak, L. Profound consequences of hidradenitis suppurativa: a review. Br. J. Dermatol. (2018). doi:10.1111/bjd.16603
3. Revuz, J. E. et al. Prevalence and factors associated with hidradenitis suppurativa: results from two case-control studies. J. Am. Acad. Dermatol. 59, 596-601 (2008).
4. Chen, W. & Plewig, G. Should hidradenitis suppurativa/acne inversa best be renamed as ‘dissecting terminal hair folliculitis’? Exp. Dermatol. 26, 544-547 (2017).
5. van der Zee, H. H. et al. Alterations in leucocyte subsets and histomorphology in normal-appearing perilesional skin and early and chronic hidradenitis suppurativa lesions. Br. J. Dermatol. 166, 98-106 (2012).
6. Paus, R. & Cotsarelis, G. The biology of hair follicles. N. Engl. J. Med. 341, 491-497 (1999).
7. Hotz, C. et al. Intrinsic defect in keratinocyte function leads to inflammation in hidradenitis suppurativa. J. Invest. Dermatol. 136, 1768-1780 (2016).
8. Barber, G. N. STING: infection, inflammation and cancer. Nat. Rev. Immunol. 15, 760-770 (2015).
9. Crow, Y. J. & Manel, N. Aicardi-Goutières syndrome and the type I interferonopathies. Nat. Rev. Immunol. 15, 429-440 (2015).
10. Coquel, F. et al. SAMHD1 acts at stalled replication forks to prevent interferon induction. Nature 557, 57-61 (2018).
11. Coquel, F., Neumayer, C., Lin, Y.-L. & Pasero, P. SAMHD1 and the innate immune response to cytosolic DNA during DNA replication. Curr. Opin. Immunol. 56, 24-30 (2019).
12. Pasero, P. & Vindigni, A. Nucleases Acting at Stalled Forks: How to Reboot the Replication Program with a Few Shortcuts. Annu. Rev. Genet. 51, 477-499 (2017).
13. Chiliveru, S. et al. Inflammatory cytokines break down intrinsic immunological tolerance of human primary keratinocytes to cytosolic DNA. J. Immunol. 192, 2395-2404 (2014).
14. Dunphy, G. et al. Non-canonical activation of the DNA sensing adaptor STING by ATM and IFI16 mediates NF-κB signaling after nuclear DNA damage. Mol. Cell 71, 745-760.e5 (2018).
15. Rheinwald, J. G. & Green, H. Serial cultivation of strains of human epidermal keratinocytes: the formation of keratinizing colonies from single cells. Cell 6, 331-343 (1975).
16. Jackson, D. A. & Pombo, A. Replicon clusters are stable units of chromosome structure: evidence that nuclear organization contributes to the efficient activation and propagation of S phase in human cells. J. Cell Biol. 140, 1285-1295 (1998).
17. Inoue, K. et al. Differential expression of stem-cell-associated markers in human hair follicle epithelial cells. Lab. Invest. 89, 844-856 (2009).
18. Zeman, M. K. & Cimprich, K. A. Causes and consequences of replication stress. Nat. Cell Biol. 16, 2-9 (2014).
19. Quinet, A., Carvajal-Maldonado, D., Lemacon, D. & Vindigni, A. DNA fiber analysis: Mind the gap! Meth. Enzymol. 591, 55-82 (2017).
20. Li, T. & Chen, Z. J. The cGAS-cGAMP-STING pathway connects DNA damage to inflammation, senescence, and cancer. J. Exp. Med. 215, 1287-1299 (2018).
21. The biology of hair follicles.-PubMed-NCBI. Available at: https://www-ncbi-nlm-nih-gov.gate2.inist.fr/pubmed/10441606. (Accessed: 3rd Apr. 2019)
22. Garza, L. A. et al. Bald scalp in men with androgenetic alopecia retains hair follicle stem cells but lacks CD200-rich and CD34-positive hair follicle progenitor cells. J. Clin.

Invest. 121, 613-622 (2011).
23. Ito, M. et al. Stem cells in the hair follicle bulge contribute to wound repair but not to homeostasis of the epidermis. Nat. Med. 11, 1351-1354 (2005).
24. Blanpain, C., Mohrin, M., Sotiropoulou, P. A. & Passeguè, E. DNA-damage response in tissue-specific and cancer stem cells. Cell Stem Cell 8, 16-29 (2011).
25. Sotiropoulou, P. A. et al. Bcl-2 and accelerated DNA repair mediates resistance of hair follicle bulge stem cells to DNA-damage-induced cell death. Nat. Cell Biol. 12, 572-582 (2010).
26. Harding, S. M. et al. Mitotic progression following DNA damage enables pattern recognition within micronuclei. Nature 548, 466-470 (2017).
27. Mackenzie, K. J. et al. cGAS surveillance of micronuclei links genome instability to innate immunity. Nature 548, 461-465 (2017).
28. King, K. R. et al. IRF3 and type I interferons fuel a fatal response to myocardial infarction. Nat. Med. 23, 1481-1487 (2017).
29. Benmerzoug, S. et al. STING-dependent sensing of self-DNA drives silica-induced lung inflammation. Nat Commun 9, 5226 (2018).
30. Almine, J. F. et al. IFI16 and cGAS cooperate in the activation of STING during DNA sensing in human keratinocytes. Nat Commun 8, 14392 (2017).
31. Wang, B. et al. Gamma-secretase gene mutations in familial acne inversa. Science 330, 1065 (2010).
32. Xiao, X. et al. Nicastrin mutations in familial acne inversa impact keratinocyte proliferation and differentiation through the Notch and phosphoinositide 3-kinase/AKT signalling pathways. Br. J. Dermatol. 174, 522-532 (2016).
33. Cao, L., Morales-Heil, D. J. & Roberson, E. D. O. Nicastrin haploinsufficiency alters expression of type I interferon-stimulated genes: the relationship to familial hidradenitis suppurativa. Clin. Exp. Dermatol. (2019). doi:10.1111/ced.13906
34. Aubin-Houzelstein, G. Notch signaling and the developing hair follicle. Adv. Exp. Med. Biol. 727, 142-160 (2012).
35. Gauntner, T. D. Hormonal, stem cell and Notch signalling as possible mechanisms of disease in hidradenitis suppurativa: a systems-level transcriptomic analysis. Br. J. Dermatol. 180, 203-204 (2019).
36. Hoffman, L. K. et al. Integrating the skin and blood transcriptomes and serum proteome in hidradenitis suppurativa reveals complement dysregulation and a plasma cell signature. PLoS ONE 13, e0203672 (2018).
37. Shanmugam, V. K., Jones, D., McNish, S., Bendall, M. L. & Crandall, K. A. Transcriptome patterns in hidradenitis suppurativa: Support for the role of antimicrobial peptides and interferon pathways in disease pathogenesis. Clin. Exp. Dermatol. (2019). doi:10.1111/ced.13959.

1. A method of treating hidradenitis suppurativa (HS) in a patient in need thereof comprising administering to the patient a therapeutically effective amount of an inhibitor of the STING pathway.

2. The method of claim 1 wherein the patient is characterized by presence of HF-SC (hair-follicle stem cells) replication stress.

3. The method of claim 2 wherein hair-follicle stem cells of the patient are characterized by the presence of at least one of the following three criteria: accumulation of cells in S phase (>25%), impaired replication fork progression, and increased proportion of cells with γ-H2AX foci (>9%).

4. The method of claim 1 wherein the inhibitor is an inhibitor of expression.

5. The method of claim 1 wherein the inhibitor is a small molecule.

6. The method of claim 5 wherein the inhibitor is N-(4-iodophenyl)-5-nitrofuran-2-carboxamide.

7. The method of claim 5 wherein the inhibitor is N-(4-Ethylphenyl)-N′-1H-indol-3-yl-urea.

8. The method of claim 1 wherein the inhibitor is used or applied on lesion area(s) of the skin.

9. The method of claim 1 wherein the inhibitor is administered in the form of a topical formulation.


SEQUENCE LISTING (1 sequence)

SEQ ID NO: 1 - DNA, 21 bp, Artificial
  siSTING
cugcauccauccaucccgutt